(19) United States
(12) Patent Application Publication
(10) Pub. No.: US 2012/0045396 A1
(43) Pub. Date: Feb. 23, 2012

(54) POROUS STRUCTURES WITH MODIFIED BIODEGRADATION KINETICS

(75) Inventors: Biana Godin-Vilentchouk, Houston, TX (US); Mauro Ferrari, Houston, TX (US)

(73) Assignee: BOARD OF REGENTS OF THE UNIVERSITY OF TEXAS SYSTEM, Austin, TX (US)

(21) Appl. No.: 13/144,724

(22) PCT Filed: Jan. 15, 2009
(86) PCT No.: PCT/US09/00239

§ 371 Date: Sep. 30, 2011

(51) Int. Cl. A61K 49/00; C07F 7/10; A61K 47/24; A61M 5/00; C08G 65/00; B82Y 5/00; B82Y 30/00

(52) U.S. Cl. 424/9.1; 604/500; 556/411; 525/474; 514/772; 977/773; 977/906; 977/927

(57) ABSTRACT

Biodegradation kinetics of biodegradable porous objects, such as porous silicon objects, can be controlled by a molecular weight of polymer chains, such as polyethylene glycol chains, disposed on an outer surface of the object. Provided are biodegradable porous objects, which have their biodegradation kinetics controlled by a molecular weight of the disposed polymer chains. Also provided are methods of making such biodegradable porous objects as well as methods of using such biodegradable porous objects for delivery of active agents, such as therapeutic agents and/or imaging agents.

### STATEMENT FOR FEDERALLY FUNDED RESEARCH

Some research underlying the invention has been supported by federal funds from NASA under grant no. SA23-06-017 and Department of Defense under grants nos. W81XWH-04-2-0035 and W81XWH-07-2-0101. The U.S. government may have certain rights in this invention.

### FIELD

The present disclosure generally relates to biodegradable structures for delivery of active agents, such as therapeutic or imaging agents and, in particular, to biodegradable porous structures, such as biodegradable porous silicon structures, for delivery of active agents and methods of making and using such structures.

### BACKGROUND

Porous silicon (pSi) was discovered by Uhlir at Bell Laboratories in the mid 1950s, ^{[1]} (a legend to the superscript citations is in the section “References”) and is currently employed in various fields of biomedical research with diverse applications including biomolecular screening, ^{[2]} optical biosensing, ^{[3, 4]} drug delivery through injectable carriers ^{[5, 6]} and implantable devices ^{[7]} as well as orally administered medications with improved bioavailability. ^{[8]} There are already several FDA approved and marketed products based on pSi technology, which found their niche in ophthalmology ^{[9]} and other, based on radioactive ³²P doped pSi is currently in clinical trials, as a potential new brachytherapy treatment for inoperable liver cancer. ^{[10]}

When porous objects, such as porous silicon objects, are used in drug delivery applications, an active agent, such as a therapeutic and/or imaging agent, can be trapped within the pores of the porous object. The release of the trapped active agent can be then achieved through a degradation of the porous over time.

Porous objects, such as porous silicon structures, were also proposed for a use in a multistage drug delivery system as larger particles (“first stage” particles), which can contain inside their pores smaller particles (“second stage particles). ^{[6]}

In a typical porous silicon drug delivery structure, the biodegradation kinetics of the porous material depends mainly on its porous properties, such as a pore size and/or porosity^{[11-13]} and, thus, is coupled to the loading capacity of the structure.

A need exists to develop a porous drug delivery system, in which the loading capacity and the biodegradation kinetics are decoupled, i.e. a system, in which the loading capacity and the biodegradation kinetics can be controlled separately from each other.

### SUMMARY

According to one embodiment, a biodegradable object comprises a porous body, that has an outer surface, and polymer chains disposed on said outer surface, wherein biodegradation kinetics of the object is determined by a pore size in the porous body and a molecular weight of the polymer chains.

According to another embodiment, a method of making a biodegradable object comprises A) obtaining an object, that has a porous body and an outer surface, wherein a biodegradation time i) is determined by a pore size of the porous body and ii) is less than a desired biodegradation time value; and B) modifying the biodegradation time of the object to the desired biodegradation time value by disposing on the outer surface of the object polymer chains, wherein the modified biodegradation time of the object is determined by the pore size of the porous body and a molecular weight of the polymer chains.

Yet according to another embodiment, a delivery method comprises introducing into a body of a subject a biodegradable object that comprises a porous body, an outer surface and polymer chains disposed on said outer surface, wherein biodegradation kinetics of the object is determined by a pore size in the porous body and a molecular weight of the polymer chains.

### BRIEF DESCRIPTION OF THE DRAWINGS

FIG. 1 schematically illustrates chemical modification of porous Si particles with APTES and PEG molecules.

FIGS. 2 (A)-(B) show graphs of degradation kinetics of large pores PEGylated Si microparticles as evaluated by ICP-AES. The degradation kinetic profile is expressed as a percentage of the total Si contents released to the degradation medium: (A) PBS pH 7.2; (B) Fetal Bovine Serum (FBS).

FIGS. 3(A)-(C) SEM images of Si particles during the degradation process in PBS pH 7.2. Systems shown: A) APTES particles; B) Particles modified with PEG 861; C) Particles modified with PEG 5000. Timepoints: 2, 8, 18 and 48 hours.

FIGS. 4(A)-(B) show graphs of erosion of fluorescent PEG vs low MW probe from the particle surface as followed up by fluorimetry in the degradation medium: (A) PBS; (B) FBS.

FIGS. 5(A)-(B) show SEM images of internalization of PEGylated (5000 D) and non-PEGylated oxidized porous silicon particles by J744 macrophages.

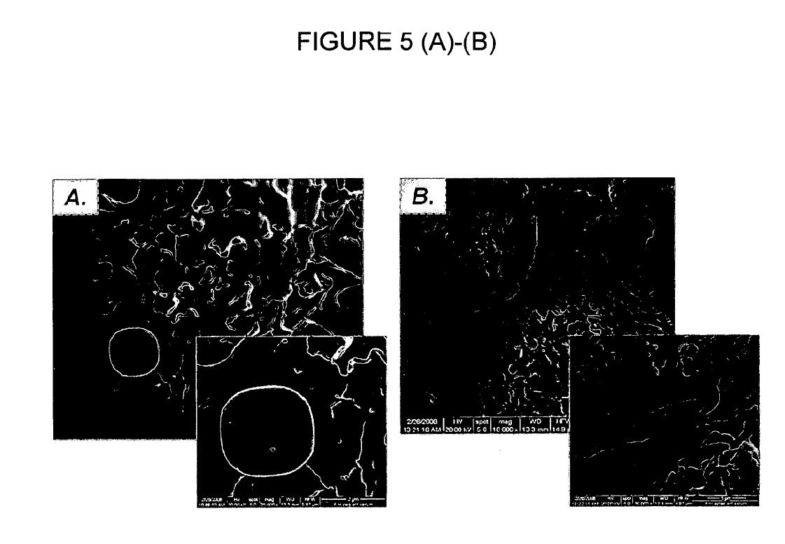

FIGS. 6 (A)-(B) demonstrate release of proinflammatory cytokines (A) IL-6 and (B) IL-8 by HUVEC cells following incubation with PEGylated and non-PEGylated particles.

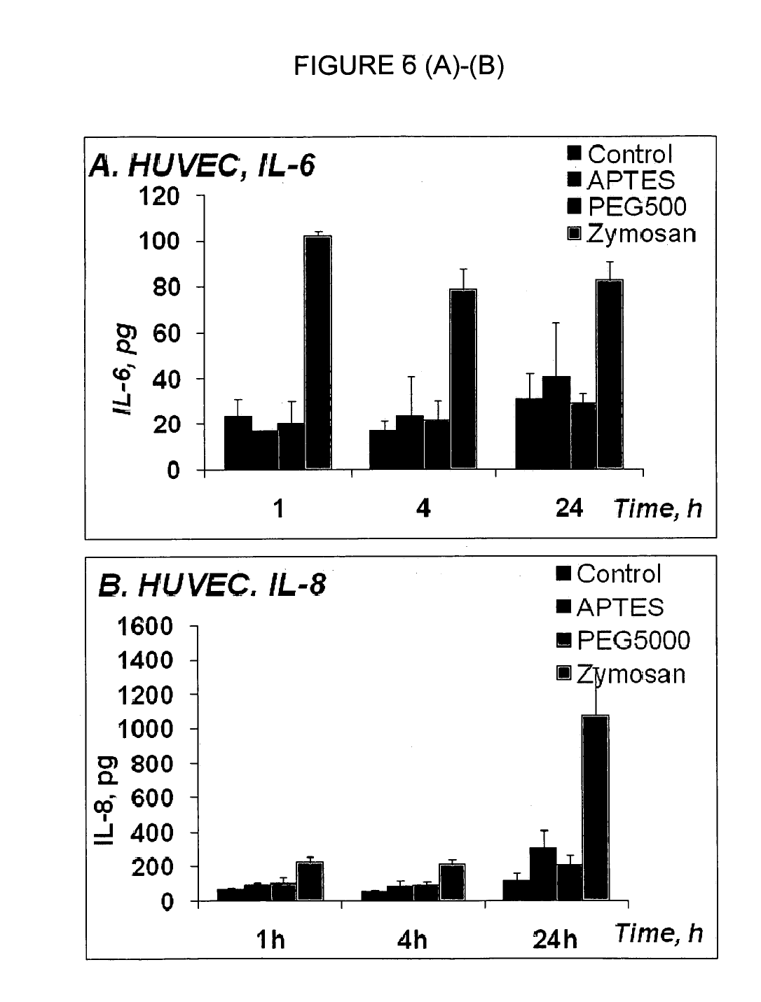

FIGS. 7(A)-(C) show effect of PMA concentration on differentiation of THP-1 monocytes into macrophages (FIG. 7A). Release of proinflammatry cytokines IL-6 (FIG. 7B) and IL-8 (FIG. 7C) by differentiated THP-1 cells following incubation with porous Si particles with various surface modifications.

FIGS. 8 (A)-(B) relates to degradation of porous silicon particles with large (30-40 nm) and small (10 nm) pores in PBS, pH 7.2 over time: (A) SEM images of particles degradation and (B) ICP data.

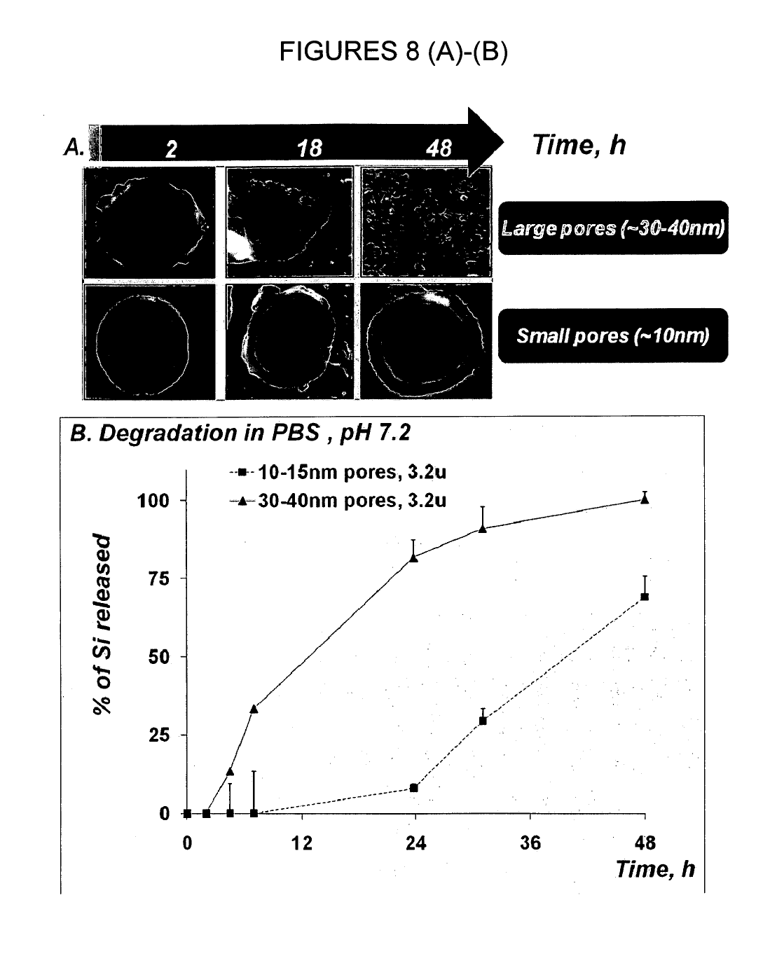

### DETAILED DESCRIPTION

### Related Applications

The following research articles and patent documents, which are all incorporated herein by reference in their entirety, may be useful for understanding the present inventions:
1) PCT publication no. WO 2007/120248 published Oct. 25, 2007;
2) PCT publication no. WO 2008/041970 published Apr. 10, 2008;
3) PCT publication no. WO 2008/021908 published Feb. 21, 2008;
4) US Patent Application Publication no. 2008/0102030 published May 1, 2008;
5) US Patent Application Publication no. 2003/0114366 published Jun. 19, 2003;
6) US Patent Application Publication no. 2008/0206344 published Aug. 28, 2008;
7) US Patent Application Publication no. 2008/0280140 published Nov. 13, 2008;
8) PCT Patent Application PCT/US2008/014001 filed Dec. 23, 2008;
9) Tasciotti E. et al, 2008 Nature Nanotechnology 3, 151-157.

### Definitions

Unless otherwise specified “a” or “an” means one or more.

“Microparticle” means a particle having a maximum characteristic size from 1 micron to 1000 microns, or from 1 micron to 100 microns. “Nanoparticle” means a particle having a maximum characteristic size of less than 1 micron.

“Nanoporous” or “nanopores” refers to pores with an average size of less than 1 micron.

“Biodegradable material” refers to a material that can dissolve or degrade in a physiological medium, such as PBS or serum.

“Biocompatible” refers to a material that, when exposed to living cells, will support an appropriate cellular activity of the cells without causing an undesirable effect in the cells such as a change in a living cycle of the cells; a release of proinflammatory factors; a change in a proliferation rate of the cells and a cytotoxic effect.

APTES stands for 3-aminopropyltriethoxysilane.

PEG refers to polyethylene glycol.

ICP-AES stands for Inductively Coupled Plasma-Atomic Emission Spectroscopy.

PBS stands for phosphate buffered saline.

FBS stands for fetal bovine serum.

SEM stands for scanning electron microscope.

HUVEC stands for Human Umbilical Vein Endothelial Cells.

PMA stands for phorbol myristate acetate.

MW stands for molecular weight.

Biodegradation kinetics refers to a time course of a biodegradation process. Biodegradation kinetics of a biodegradable object can depend on a particular physiological medium, in which the biodegradation process takes place. A comparison between biodegradation kinetics for different objects should be made with respect to the same physiological medium.

Biodegradation kinetics can be represented graphically as a biodegradation kinetic profile.

Biodegradation time refers to a time it requires for a biodegradable object to fully degrade in a particular physiological medium.

Loading capacity or loading efficiency refers to an amount of a load that can be contained in pores of a porous object.

Physiological conditions stand for conditions, such as the temperature, osmolarity, pH and motion close, close to that of plasma in a mammal body, such as a human body, in the normal state.

### Disclosure

The present inventors discovered that a surface modification of a porous biodegradable object, such as a porous implant or a porous particle, can be used for controlling biodegradation kinetics of the object. Thus, the object's biodegradation kinetics may be decoupled from the object's porous properties, i.e. porosity and/or pore size, and thus from the object's loading capacity. In other words, one can modify the biodegradation kinetics of the object without substantially changing the loading capacity of the object.

The surface modification can refer to a modification of an outer surface of the object. The surface modification can be performed by disposing on an outer surface of the biodegradable porous object polymer chains, such as hydrophilic polymer chains.

Thus, one embodiment can be a biodegradable porous object, such as a porous implant or a porous particle, that can have a biodegradation kinetics, which is different from a biodegradation kinetics determined by its porous properties.

The biodegradable porous object can comprise a porous body, that has an outer surface, and polymer chains, preferably hydrophilic polymer chains, that are disposed on the outer surface. The object can be such that its biodegradation kinetics is effectively determined by a pore size (or porosity) of the porous body and a molecular weight of the polymer chains disposed on the object. In other words, the molecular weight of the polymer chains is such that the disposed polymer chains modify the biodegradation kinetics of the object compared a biodegradation kinetics of the otherwise analogous porous object, that does not have the polymer chains disposed.

Another embodiment can be a method of making a biodegradable object that has a desired biodegradation kinetics or time. Such a method can involve selecting a desired biodegradation time or kinetics; obtaining an initial porous object, which has its biodegradation time determined by its porous properties, i.e. its pore size and/or porosity (this degradation time is less than the desired biodegradation time); and disposing polymer chains on the outer surface of the object and, thereby, modifying the biodegradation time object to the desired value. The modified biodegradation time can be effectively determined by a combination of the porous properties of the porous body, i.e. a pore size and/or porosity, and a molecular weight of the disposed polymer chains.

### Modified Biodegradation Kinetics

Surface modification, such as deposition of polymer chains, can impede the biodegradation of the biodegradable porous object, i.e. increase a biodegradation time of the object compared to an otherwise analogous biodegradable porous object without the surface modification. For example, for a porous silicon object having an average pore size from 5 to 200 nm or from 5 to 150 nm or from 5 to 120 nm or from 10 to 100 nm or 10 to 80 nm or from 20 to 70 nm or 25 to 60 nm or from 30 nm to 50 nm or any integer within these ranges a biodegradation time in physiological conditions can be at least 24 hours or at least 36 hours or at least 48 hours or at least 60 hours or at least 72 hours or at least 84 hours or at least 96 hours or at least 108 hours or at least 120 hours or at least 132 hours or at least 144 hours or at least 156 hours or at least 168 hours or at least 180 hours or at least 192 hours.

Surface modification, such as deposition of polymer chains, can produce a heterogeneous biodegradation profile can include a first time period and a second time period, such that during the first time period the degraded material is released at a rate, which is different from a rate at which the degraded material is released during the second period. The heterogeneous profile can include more than two time periods with different release rates. In some cases, the heterogeneous profile can include a) a first period, which starts when the biodegradable object is introduced into a physiological medium, such that no degradation or substantially no degradation occurs during it; b) a second period, during which a substantial degradation of the object occurs. In some embodiments, the first period, when no degradation or substantially no degradation occurs, for a porous silicon object having an average pore size from 5 to 200 nm or from 5 to 150 nm or from 5 to 120 nm or from 10 to 100 nm or 10 to 80 nm or from 20 to 70 nm or 25 to 60 nm or from 30 nm to 50 nm or any integer within these ranges, in physiological conditions can be at least 6 hours or at least 12 hours or at least 15 hours or at least 18 hours.

### Polymer Chains

The polymer chains disposed on the outer surface of the biodegradable porous object are preferably hydrophilic polymer chains, such as polyethylene glycols (PEGs) or synthetic glycocalix chains. In the prior art, PEGs are mainly used in classical drug delivery systems, i.e. non-porous systems, and in pharmaceutical dosage forms, to avoid reticulo-endothelial system (RES) uptake and thus to control biodistribution and circulation time. ^{[13]} PEGs are approved by FDA for use in food, cosmetics and pharmaceuticals, including injectable, topical, rectal and nasal formulations. PEG molecules demonstrate little toxicity, and are cleared from the body, without being metabolized, by either the kidneys for PEGs<30 kDa or in the feces for longer PEGs.

Heavier molecular weight polymer chains can affect the biodegradation of the porous biodegradable stronger than lower molecular weight. Particular values of polymer chains' molecular weight, for which the disposed chains start effectively modifying the biodegradation kinetics of the biodegradable porous object can depend on a number of factors including a pore size of the porous object. For example, for porous silicon objects, having an average pore size, ranging from 25 to 60 nm, polymer chains that can modify the biodegradation kinetics when disposed on the object, have molecular weight of no less than 400, or no less than 800, or from 800 to 30,000 or 800 to 20,000 or from 800 to 10,000 or from 800 to 7000 or from 1000 to 6000 or from 2000 to 6000 or from 3000 to 6000 or any integer between these ranges.

Polymer chains can be covalently attached to an outer surface of the biodegradable porous object. When the object's surface material comprises an oxide, such as silicon oxide in a case of a porous silicon biodegradable object, the polymer chains can be attached using silane chemistry. For example, first an aminosilane, such as 3-aminopropyltriethoxysilane (APTES), can be deposited on the outer surface, then a succinimidyl-ester (SCM) terminated polymer chain can be coupled to the amine group of the aminosilane. Coupling chemistries, other than SCM-amine, can be also used for covalent attachment of polymer chains.

### Targeting Moieties

When the porous object is a porous particle, its outer surface can comprise one or more targeting moities, such as a dendrimer, an antibody, an aptamer, which can be a thioaptamer, a ligand, such as an E-selectin or P-selectin, or a biomolecule, such as an RGD peptide. The targeting moieties can be used to target and/or localize the particle a specific site in a body of a subject. The targeted site can be a vasculature site. In some embodiments, the vasculature site can be a tumor vasculature, such as angiogenesis vasculature, coopted vasculature or renormalized vasculature.

The selectivity of the targeting can be tuned by changing chemical moieties of the surface of the particles. For example, coopted vasculature can be specifically using antibodies to angiopoietin 2; angiogenic vasculature can be recognized using antibodies to vascular endothelial growth factor (VEGF), basic fibroblast growth factor (FGFb) or endothelial markers such as αᵥβ₃ integrins, while renormalized vasculature can be recognized using carcinoembionic antigen-related vell adhesion molecule 1 (CEACAM1), endothelin-B receptor (ET-B), vascular endothelial growth factor inhibitors gravin/AKAP12, a scallofldoing protein for protein kinase A and protein kinase C, see e.g. Robert S. Korbel “Antiangiogenic Therapy: A Universal Chemosensitization Strategy for Cancer?”, Science 26 May 2006, vol 312, no. 5777, 1171-1175.

In some embodiments, the targeting moieties can be attached covalently or non-covalently directly to the surface of the particle. Yet in some embodiments, the targeting moieties can be can be attached covalently or non-covalently to the polymer chains disposed on the outer surface of the particle.

### Porous Object

The porous object can be a porous implant or a porous particle.

The porous implant can have a variety of shapes and sizes. The dimensions of the porous implant are not particularly limited and depend on an application. In some embodiments, the porous implant can have a minimal dimension of no less than 0.1 mm or no less than 0.2 mm no less than 0.2 mm or no less than 0.3 mm or no less than 0.5 mm or no less than 1.0 mm or no less than 2 mm or no less than 5 mm or no less than 10 mm or no less than 20 mm. In some embodiments, the porous implant can have at least two dimensions of no less than 0.1 mm or no less than 0.2 mm no less than 0.2 mm or no less than 0.3 mm or no less than 0.5 mm or no less than 1.0 mm or no less than 2 mm or no less than 5 mm or no less than 10 mm or no less than 20 mm. Porous silicon implants are disclosed, for example, in WO99/53898, which is incorporated herein in its entirety.

The porous particle can also have a variety of shapes and sizes. The dimensions of the porous particle are not particularly limited and depend on an application. For example, for intravascular administration, a maximum characteristic size of the particle can be smaller than a radius of the smallest capillary in a subject, which is about 4 to 5 microns for humans. In some embodiments, the maximum characteristic size of the porous particle may be less than about 100 microns or less than about 50 microns or less than about 20 microns or less than about 10 microns or less than about 5 microns or less than about 4 microns or less than about 3 microns or less than about 2 microns or less than about 1 micron. Yet in some embodiments, the maximum characteristic size of the porous particle may be from 100 nm to 3 microns or from 200 nm to 3 microns or from 500 nm to 3 microns or from 700 nm to 2 microns.

Yet in some embodiments, the maximum characteristic size of the porous particle may be greater than about 2 microns or greater than about 5 microns or greater than about 10 microns.

The shape of the porous particle is not particularly limited. In some embodiments, the particle can be a spherical particle. Yet in some embodiments, the particle can be a non-spherical particle. In some embodiments, the particle can have a symmetrical shape. Yet in some embodiments, the particle can have an asymmetrical shape.

In some embodiments, the particle can have a selected non-spherical shape configured to facilitate a contact between the particle and a surface of the target site, such as endothelium surface of the inflamed vasculature. Examples of appropriate shapes include, but not limited to, an oblate spheroid, a disc or a cylinder. In some embodiments, the particle can be such that only a portion of its outer surface defines a shape configured to facilitate a contact between the particle and a surface of the target site, such as endothelium surface, while the rest of the outer surface does not. For example, the particle can be a truncated oblate spheroidal particle.

The dimensions and shape of particle that can facilitate a contact between the particle and a surface of the target site can be evaluated using methods disclosed in US Patent Application Publication no. 2008/0206344 and U.S. Application no. 12/181,759 filed Jul. 29, 2008.

### Porous Material

The porous object, such as an implant or a particle, comprises a porous material. In many embodiments, the porous material can be a non-polymer porous material such as a porous oxide material or a porous etched material. Examples of porous oxide materials include, but no limited to, porous silicon oxide, porous aluminum oxide, porous titanium oxide and porous iron oxide. The term “porous etched materials” refers to a material, in which pores are introduced via a wet etching technique, such as electrochemical etching. Examples of porous etched materials include porous semiconductors materials, such as porous silicon, porous germanium, porous GaAs, porous InP, porous SiC, porous SiₓGe₁₋ₓ, porous GaP, porous GaN. Methods of making porous etched particles are disclosed, for example, US Patent Application Publication no. 2008/0280140.

In many embodiments, the porous object can be a nanoporous object.

In some embodiments, a average pore size of the porous object may be from about 1 nm to about 1 micron or from about 1 nm to about 800 nm or from about 1 nm to about 500 nm or from about 1 nm to about 300 nm or from about 1 nm to about 200 nm or from about 2 nm to about 100 nm or any integer within these ranges.

In some embodiments, the average pore size of the porous object can be no more than 1 micron or no more than 800 nm or more than 500 nm or more than 300 nm or no more than 200 nm or no more than 100 nm or no more than 80 nm or no more than 50 nm. In some embodiments, the average pore size of the porous object can be size from about from 5 to 200 nm or from 5 to 150 nm or from 5 to 120 nm or from 10 to 100 nm or 10 to 80 nm or from 20 to 70 nm or 25 to 60 nm or from 30 nm to 50 nm or any integer within these ranges.

In some embodiments, the average pore size of the porous particle can be from about 3 nm to about 10 nm or from about 3 nm to about 10 nm or from about 3 nm to about 7 nm or any integer between these ranges.

In general, pores sizes may be determined using a number of techniques including N₂ adsorption/desorption and microscopy, such as scanning electron microscopy.

In some embodiments, pores of the porous particle may be linear pores. Yet in some embodiments, pores of the porous particle may be sponge like pores.

Porous silicon particles and methods of their fabrication are disclosed, for example, in Cohen M. H. et al Biomedical Microdevices 5:3, 253-259, 2003; US patent application publication no. 2003/0114366; U.S. Pat. Nos. 6,107,102 and 6,355,270; US Patent Application Publication no. 2008/0280140; PCT publication no. WO 2008/021908; Foraker, A. B. et al. Pharma. Res. 20 (1), 110-116 (2003); Salonen, J. et al. Jour. Contr. Rel. 108, 362-374 (2005). Porous silicon oxide particles and methods of their fabrication are disclosed, for example, in Paik J. A. et al. J. Mater. Res., Vol 17, Aug 2002, p. 2121.

### Fabrication

The porous objects, such as porous implants or porous particles, may be prepared using a number of techniques.

For example, in some embodiments, the porous objects may be a top-down fabricated object, i.e. a object produced utilizing a top-down microfabrication or nanofabrication technique, such as photolithography, electron beam lithography, X-ray lithography, deep UV lithography, nanoimprint lithography or dip pen nanolithography. Such fabrication methods may allow for a scaled up production of porous particles, that are uniform or substantially identical in dimensions.

### Biocompatibility

The biodegradable porous objects with modified biodegradation kinetics can be biocompatible. In particular, the biodegradable porous objects with modified biodegradation kinetics can be such that they do not induce release of proinflamatory cytokines, such IL-6 and IL-8 during the biodegradation.

### Loading

Active agents and/or smaller particles can be loaded into pores of the biodegradable porous objects using a number of methods including those disclosed in US patent applications nos. US2008280140 and 20030114366; in PCT publications nos. WO20080219082 and WO 99/53898.

### Applications

The biodegradable porous objects with modified biodegradation kinetics can be used for pharmaceutical, cosmetic, medical, veterinary, diagnostic and research applications. For example, the biodegradable porous objects can be used for delivering an active agent, such as a therapeutic agent and/or an imaging agent, when introduced in a body of a subject, which can be, for example, a mammal, such as a human being. Thus, the biodegradable objects can be used for treating, preventing or monitoring a disease or a condition in the subject. Particular diseases/conditions can depend on particular active agents. Non-limiting examples of diseases/conditions include cancer and inflammation, neurodegenerative disorders, skin disorders, cardiovascular conditions, endocrinological disorders, pregnancy, diabetes, infectious (such as microbial, parasite, fungal) diseases.

In some embodiments, the active agent can be contained within pores of the porous body. For example, the active agent can be a chemical molecule trapped within the pores via a specific and/or non specific interactions.

In some embodiments, pores of the biodegradable porous object can contain smaller size particles, which can contain an active agent. In such a case, the biodegradable porous object can be a part of a multistage drug delivery system, such as the types which are disclosed, for example, in US patent application no. US2008280140 and in PCT publication no. WO2008021908.

In some embodiments, the porous body of the porous object can contain the active agent. For example, the porous body of the porous object can be made of a radioactive material. Such a radioactive porous object can be used for radiotherapy treatment of cancer, such as breast cancer, prostate cancer, cervical cancer, liver cancer, lymphoma, ovarian cancer and melanoma. One non-limiting example of radioactive porous material can be porous silicon doped with radioactive ³²P.

### Active Agent

The active agent can be a therapeutic agent, an imaging agent or a combination thereof. The selection of the active agent depends on a particular application.

### Therapeutic Agent

The therapeutic agent may be any physiologically or pharmacologically active substance that can produce a desired biological effect in a targeted site in an animal, such as a mammal or a human. The therapeutic agent may be any inorganic or organic compound, without limitation, including peptides, proteins, nucleic acids including siRNA, miRNA and DNA, polymers and small molecules, any of which may be characterized or uncharacterized. The therapeutic agent may be in various forms, such as an unchanged molecules, molecular complex, pharmacologically acceptable salt, such as hydrochloride, hydrobromide, sulfate, laurate, palmitate, phosphate, nitrite, nitrate, borate, acetate, maleate, tartrate, oleate, salicylate, and the like. For acidic therapeutic agent, salts of metals, amines or organic cations, for example, quaternary ammonium, can be used. Derivatives of drugs, such as bases, esters and amides also can be used as a therapeutic agent. A therapeutic agent that is water insoluble can be used in a form that is a water soluble derivative thereof, or as a base derivative thereof, which in either instance, or by its delivery, is converted by enzymes, hydrolyzed by the body pH, or by other metabolic processes to the original therapeutically active form.

Examples of therapeutic agents include, but are not limited to, anti-cancer agents, such as anti-proliferative agents, anti-vascularization agents; antimalarial agents; OTC drugs, such as antipyretics, anesthetics, cough suppressants; antiinfective agents; antiparasites, such as anti-malaria agents such as Dihydroartemisin; antibiotics, such as penicillins, cephalosporins, macrolids, tetracyclines, aminglycosides, anti-tuberculosis agents; antifungal/antimycotic agent; genetic molecules, such as anti-sense oligonucleotides, nucleic acids, oligonucleotides, DNA, RNA; anti-protozoal agents; antiviral agents, such as acyclovir, gancyclovir, ribavirin, anti-HIV agents, anti-hepatitis agents; anti-inflammatory agents, such as NSAIDs, steroidal agents, cannabinoids; anti-allergic agents, such as antihistamines, fexofenadine);

bronchodilators; vaccines or immunogenic agents, such as tetanus toxoid, reduced diphtheria toxoid, acellular pertussis vaccine, mums vaccine, smallpox vaccine, anti-HIV vaccines, hepatitis vaccines, pneumonia vaccines, influenza vaccines; anesthetics including local anesthetics; antipyretics, such as paracetamol, ibuprofen, diclofenac, aspirin; agents for treatment of severe events such as cardiovascular attacks, seizures, hypoglycemia; anti-nausea and anti-vomiting agents; immunomodulators and immunostimulators; cardiovascular drugs, such as beta-blockers, alpha-blockers, calcium channel blockers; peptide and steroid hormones, such as insulin, insulin derivatives, insulin detemir, insulin monomeric, oxytocin, LHRH, LHRH analogues, adreno-corticotropic hormone, somatropin, leuprolide, calcitonin, parathyroid hormone, estrogens, testosterone, adrenal corticosteroids, megestrol, progesterone, sex hormones, growth hormones, growth factors; peptide and protein related drugs, such as amino acids, peptides, polypeptides, proteins; vitamins, such as Vitamin A, Vitamins from B group, folic acid, Vit C, Vit D, Vit E, Vit K, niacin, derivatives of Vit D; Autonomic Nervous System Drugs; fertilizing agents; antidepressants, such as buspirone, venlafaxine, benzodiazepins, selective serotonin reuptake inhibitors (SSRIs), sertraline, citalopram, tricyclic antidepressants, paroxetine, trazodone, lithium, bupropion, sertraline, fluoxetine; agents for smoking cessation, such as bupropion, nicotine; lipid-lowering agents, such as inhibitors of 3 hydroxy-3-methylglutaryl-coenzyme A (HMG-CoA) reductase, simvastatin, atrovastatinl; agents for CNS or spinal cord, such as benzodiazepines, lorazepam, hydromorphone, midazolam, Acetaminophen, 4′-hydroxyacetanilide, barbiturates, anesthetics; anti-epilepsic agents, such as valproic acid and its derivatives, carbamazepin; angiotensin antagonists, such as valsartan; anti-psychotic agents and anti-schizophrenic agents, such as quetiapine, risperidone; agents for treatment of Parkinsonian syndrome, such as L-dopa and its derivatives, trihexyphenidyl; anti-Alzheimer agents, such as cholinesterase inhibitors, galantamine, rivastigmine, donepezil, tacrine, memantine, N-methyl D-aspartate (NMDA) antagonists; agents for treatment of non-insulin dependent diabetes, such as metformin, agents against erectile dysfunction, such as sildenafil, tadalafil, papaverine, vardenafil, PGE1; prostaglandins; agents for bladder dysfunction, such as oxybutynin, propantheline bromide, trospium, solifenacin succinate; agents for treatment menopausal syndrome, such as estrogens, non-estrogen compounds, agents for treatment hot flashes in postmenopausal women; agents for treatment primary or secondary hypogonadism, such as testosterone; cytokines, such as TNF, interferons, IFN-alpha, IFN-beta, interleukins; CNS stimulants; muscle relaxants; anti paralytic gas agents; narcotics and Antagonists, such as opiates, oxycodone; painkillers, such as opiates, endorphins, tramadol, codein, NSAIDs, gabapentine; Hypnotics, such as Zolpidem, benzodiazepins, barbiturates, ramelteon; Histamines and Antihistamines; Antimigraine Drugs such as imipramine, propranolol, sumatriptan; diagnostic agents, such as Phenolsulfonphthalein, Dye T-1824, Vital Dyes, Potassium Ferrocyanide, Secretin, Pentagastrin, Cerulein; topical decongestants or anti-inflammatory agents; anti-acne agents, such as retinoic acid derivatives, doxicillin, minocyclin; ADHD related agents, such as methylphenidate, dexmethylphenidate, dextroamphetamine, d- and l-amphetamin racemic mixture, pemoline; diuretic agents; anti-osteoporotic agents, such as. bisphosphonates, aledronate, pamidronate, tirphostins; osteogenic agents; anti-asthma agents; anti-Spasmotic agents, such as papaverine; agents for treatment of multiple sclerosis and other neurodegenerative disorders, such as mitoxantrone, glatiramer acetate, interferon beta-1a, interferon beta-1b; plant derived agents from leave, root, flower, seed, stem or branches extracts.

The therapeutic agent can be a chemotherapeutic agent, an immunosuppressive agent, a cytokine, a cytotoxic agent, a nucleolytic compound, a radioactive isotope, a receptor, and a pro-drug activating enzyme, which may be naturally occurring or produced by synthetic or recombinant methods, or any combination thereof.

Drugs that are affected by classical multidrug resistance, such as vinca alkaloids (e.g., vinblastine and vincristine), the anthracyclines (e.g., doxorubicin and daunorubicin), RNA transcription inhibitors (e.g., actinomycin-D) and microtubule stabilizing drugs (e.g., paclitaxel) can have particular utility as the therapeutic agent.

A cancer chemotherapy agent may be a preferred therapeutic agent. Useful cancer chemotherapy drugs include nitrogen mustards, nitrosorueas, ethyleneimine, alkane sulfonates, tetrazine, platinum compounds, pyrimidine analogs, purine analogs, antimetabolites, folate analogs, anthracyclines, taxanes, vinca alkaloids, topoisomerase inhibitors and hormonal agents. Exemplary chemotherapy drugs are Actinomycin-D, Alkeran, Ara-C, Anastrozole, Asparaginase, BiCNU, Bicalutamide, Bleomycin, Busulfan, Capecitabine, Carboplatin, Carboplatinum, Carmustine, CCNU, Chlorambucil, Cisplatin, Cladribine, CPT-11, Cyclophosphamide, Cytarabine, Cytosine arabinoside, Cytoxan, Dacarbazine, Dactinomycin, Daunorubicin, Dexrazoxane, Docetaxel, Doxorubicin, DTIC, Epirubicin, Ethyleneimine, Etoposide, Floxuridine, Fludarabine, Fluorouracil, Flutamide, Fotemustine, Gemcitabine, Herceptin, Hexamethylamine, Hydroxyurea, Idarubicin, Ifosfamide, Irinotecan, Lomustine, Mechlorethamine, Melphalan, Mercaptopurine, Methotrexate, Mitomycin, Mitotane, Mitoxantrone, Oxaliplatin, Paclitaxel, Pamidronate, Pentostatin, Plicamycin, Procarbazine, Rituximab, Steroids, Streptozocin, STI-571, Streptozocin, Tamoxifen, Temozolomide, Teniposide, Tetrazine, Thioguanine, Thiotepa, Tomudex, Topotecan, Treosulphan, Trimetrexate, Vinblastine, Vincristine, Vindesine, Vinorelbine, VP-16, and Xeloda.

Useful cancer chemotherapy drugs also include alkylating agents, such as Thiotepa and cyclosphosphamide; alkyl sulfonates such as Busulfan, Improsulfan and Piposulfan; aziridines such as Benzodopa, Carboquone, Meturedopa, and Uredopa; ethylenimines and methylamelamines including altretamine, triethylenemelamine, trietylenephosphoramide, triethylenethiophosphaoramide and trimethylolomelamine; nitrogen mustards such as Chlorambucil, Chlornaphazine, Cholophosphamide, Estramustine, Ifosfamide, mechlorethamine, mechlorethamine oxide hydrochloride, Melphalan, Novembiehin, Phenesterine, Prednimustine, Trofosfamide, uracil mustard; nitroureas such as Cannustine, Chlorozotocin, Fotemustine, Lomustine, Nimustine, and Ranimustine; antibiotics such as Aclacinomysins, Actinomycin, Authramycin, Azaserine, Bleomycins, Cactinomycin, Calicheamicin, Carabicin, Carminomycin, Carzinophilin, Chromoinycins, Dactinomycin, Daunorubicin, Detorubicin, 6-diazo-5-oxo-L-norleucine, Doxorubicin, Epirubicin, Esorubicin, Idambicin, Marcellomycin, Mitomycins, mycophenolic acid, Nogalamycin, Olivomycins, Peplomycin, Potfiromycin, Puromycin, Quelamycin, Rodorubicin, Streptonigrin, Streptozocin, Tubercidin, Ubenimex, Zinostatin, and Zorubicin; anti-metabolites such as Methotrexate and 5-fluorouracil (5-FU); folic acid analogues such as Denopterin, Methotrexate, Pteropterin, and Trimetrexate; purine analogs such as Fludarabine, 6-mercaptopurine, Thiamiprine, and Thioguanine; pyrimidine analogs such as Ancitabine, Azacitidine, 6-azauridine, Carmofur, Cytarabine, Dideoxyuridine, Doxifluridine, Enocitabine, Floxuridine, and 5-FU; androgens such as Calusterone, Dromostanolone Propionate, Epitiostanol, Rnepitiostane, and Testolactone; anti-adrenals such as aminoglutethimide, Mitotane, and Trilostane; folic acid replenisher such as frolinic acid; aceglatone; aldophosphamide glycoside; aminolevulinic acid; Amsacrine; Bestrabucil; Bisantrene; Edatraxate; Defofamine; Demecolcine; Diaziquone; Elfornithine; elliptinium acetate; Etoglucid; gallium nitrate; hydroxyurea; Lentinan; Lonidamine; Mitoguazone; Mitoxantrone; Mopidamol; Nitracrine; Pentostatin; Phenamet; Pirarubicin; podophyllinic acid; 2-ethylhydrazide; Procarbazine; PSK®; Razoxane; Sizofrran; Spirogermanium; tenuazonic acid; triaziquone; 2,2′,2″-trichlorotriethylamine; Urethan; Vindesine; Dacarbazine; Mannomustine; Mitobronitol; Mitolactol; Pipobroman; Gacytosine; Arabinoside (“Ara-C”); cyclophosphamide; thiotEPa; taxoids, e.g., Paclitaxel (TAXOL®, Bristol-Myers Squibb Oncology, Princeton, N.J.) and Doxetaxel (TAXOTERE®, Rhone-Poulenc Rorer, Antony, France); Chlorambucil; Gemcitabine; 6-thioguanine; Mercaptopurine; Methotrexate; platinum analogs such as Cisplatin and Carboplatin; Vinblastine; platinum; etoposide (VP-16); Ifosfamide; Mitomycin C; Mitoxantrone; Vincristine; Vinorelbine; Navelbine; Novantrone; Teniposide; Daunomycin; Aminopterin; Xeloda; Ibandronate; CPT-11; topoisomerase inhibitor RFS 2000; difluoromethylornithine (DMFO); retinoic acid; Esperamicins; Capecitabine; and pharmaceutically acceptable salts, acids or derivatives of any of the above. Also included are anti-hormonal agents that act to regulate or inhibit hormone action on tumors such as anti-estrogens including for example Tamoxifen, Raloxifene, aromatase inhibiting 4(5)-imidazoles, 4 Hydroxytamoxifen, Trioxifene, Keoxifene, Onapristone, And Toremifene (Fareston); and anti-androgens such as Flutamide, Nilutamide, Bicalutamide, Leuprolide, and Goserelin; and pharmaceutically acceptable salts, acids or derivatives of any of the above.

Cytokines can be also used as the therapeutic agent. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet growth factor; transforming growth factors (TGFs) such as TGF-α and TGF-β; insulin-like growth factor-I and -II; erythropoietin (EPO); osteoinductive factors; interferons such as interferon-α, -β and -γ; colony stimulating factors (CSFs) such as macrophage-CSF (M-CSF); granulocyte-macrophage-CSF (GM-CSF); and granulocyte-CSF (GCSF); interleukins (ILs) such as IL-1, IL-1a, IL-2, IL-3, IL-4, IL-5, IL-6, IL-7, IL-8, IL-9, IL-11, IL-12, IL-15; a tumor necrosis factor such as TNF-α or TNF-β; and other polypeptide factors including LIF and kit ligand (KL). As used herein, the tern cytokine includes proteins from natural sources or from recombinant cell culture and biologically active equivalents of the native sequence cytokines.

In some embodiments, the therapeutic agent can be an antibody-based therapeutic agent, such as herceptin.

In some embodiments, the therapeutic agent can be a nanoparticle. For example, in some embodiments, the nanoparticle can be a nanoparticle that can be used for a thermal oblation or a thermal therapy. Examples of such nanoparticles include iron and gold nanoparticles.

### Imaging Agent

The imaging agent can be a substance that can provide imaging information about a targeted site in a body of an animal, such as a mammal or a human being. The imaging agent can comprise a magnetic material, such as iron oxide or a gadolinium containing compound, for magnetic resonance imaging (MRI). For optical imaging, the active agent can be, for example, semiconductor nanocrystal or quantum dot. For optical coherence tomography imaging, the imaging agent can be metal, e.g. gold or silver, nanocage particles. The imaging agent can be also an ultrasound contrast agent, such as a micro or nanobubble or iron oxide micro or nanoparticle. In some embodiments, the imaging agent can a molecular imaging agent that can be covalently or non-covalently attached to a particle's surface.

### Administration

When the porous biodegradable object is a porous micro or nanoparticle(s) can be administered as a part of a composition, that includes a plurality of the particles, to a subject, such as human, via a suitable administration method in order to treat, prevent and/or monitor a physiological condition, such as a disease.

The particular method employed for a specific application can be determined by the attending physician. Typically, the composition can be administered by one of the following routes: topical, parenteral, inhalation/pulmonary, oral, intraocular, intranasal, bucal, vaginal and anal.

The particles can be particularly useful for oncological applications, i.e. for treatment and/or monitoring cancer or a condition, such as tumor associated with cancer.

The majority of therapeutic applications can involve some type of parenteral administration, which includes intravenous (i.v.), intramuscular (i.m.) and subcutaneous (s.c.) injection. Administration of the particles can be systemic or local. The non-parenteral examples of administration recited above are examples of local administration. Intravascular administration can be either local or systemic. Local intravascular delivery can be used to bring a therapeutic substance to the vicinity of a known lesion by use of guided catheter system, such as a CAT-scan guided catheter, portal vein injectionr. General injection, such as a bolus i.v. injection or continuous/trickle-feed i.v. infusion are typically systemic.

Preferably, the composition containing particles is administered via i.v. infusion, via intraductal administration or via intratumoral route.

The particles may be formulated as a suspension that contains a plurality of the particles.

Preferably, the particles are uniform in their dimensions and their content. To form the suspension, the particles as described above can be suspended in any suitable aqueous carrier vehicle. A suitable pharmaceutical carrier may the one that is non-toxic to the recipient at the dosages and concentrations employed and is compatible with other ingredients in the formulation. Preparation of suspension of microfabricated particles is disclosed, for example, in US patent application publication No. 20030114366.

Embodiments described herein are further illustrated by, though in no way limited to, the following working examples.

### WORKING EXAMPLES

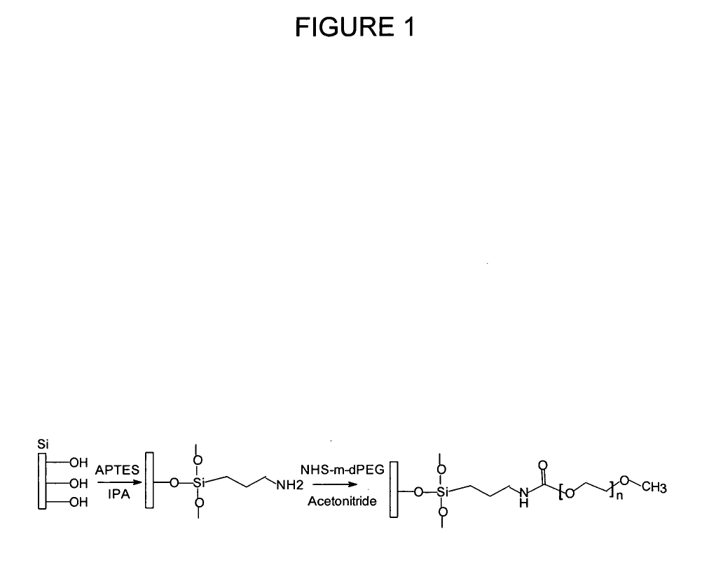

Mesoporous hemispherical silicon microparticles were fabricated by photolithography and electrochemical etching as previously described. ^{[6]} In this study, standard surface modification procedures developed for silicon-based materials were used (schematically presented in FIG. 1).

During the oxidation process, partial erosion of the particle surface led to an introduction of free hydroxyl groups, imparting to the particles a negative zeta potential (−31.5 mV). Through silane chemistry the hydroxyl surface groups were covalently coupled to positively charged 3-Aminopropyltriethoxysilane (APTES), reversing the net surface charge of the particles to +14.73 mV.

APTES amine groups further served as a background for linking molecules to the particles surface. First, to estimate the range of molar ratios suitable for further conjugation of surface modifiers, the effect of fluorescent probe concentration in the reaction medium on the fluorescence of the silicon particles was evaluated. In the concentration range of 3.75-15 mM of the 488-Dylight in the reaction medium, the net fluorescence intensity of the particles reached a plateau, which can be attributed to saturation of the bindings sites on the particles surface. A slight reduction in the fluorescence intensity of the particles was observed at higher concentrations of the probe, which could be related to the quenching effect of the probe on the surface. This general behavior was consistent and repetitive among different experiments, though numerical values of fluorescent intensity slightly vary, due to the slightly different surface area and properties of pSi microparticles. Based on these results, a concentration of 10 mM of PEG was chosen in order to obtain a saturation of the modifier on the particle surface. As in the case of the fluorescent probe, PEG molecules (MWs from 245 to 5000) were bound to the particles through APTES amine groups. No direct correlation was observed between the length of the PEG molecule and the zeta potential values (see, Table 1), though all PEGs and fluorescent probes bound to APTES amine groups resulted in a neutralization of the positive charges introduced by APTES thus causing a slightly negative zeta potential, which could be partially explained by the charge-shielding effect of PEG backbones.

TABLE 1
Description and zeta potential values of the investigated microparticles.

| | Particles zeta potential, |
|---|---|
| Sample name | mV Mean ± SD (n = 4) |
| Oxidized | −31.05 ± 2.73 |
| APTES | +14.73 ± 1.62 |
| PEG 245 | −15.58 ± 3.58 |
| PEG 333 | −3.69 ± 2.23 |
| PEG 509 | −9.68 ± 2.49 |
| PEG 686 | −13.44 ± 3.40 |
| PEG 862 | −3.94 ± 1.91 |
| PEG 1214 | −11.29 ± 1.93 |
| PEG 3400-Dylight 488 | −8.34 ± 2.51 |
| PEG 5000 | −6.48 ± 2.84 |
| Dylight 488 | −15.44 ± 3.40 |

To evaluate the degradation rate of the particles under the simulated physiological conditions, the degradation of small pores (10 nm) and large pores (30-50 nm) non-PEGylated APTES particles in phosphate buffered saline (PBS, pH 7.2) and fetal bovine serum serum (FBS) was tested initially. In agreement with the published literature, degradation kinetics of the mesoporous Si particles was strongly dependant on the pore size ^{[12]}. Particles having small pores degraded much slower than the particles with large pores.

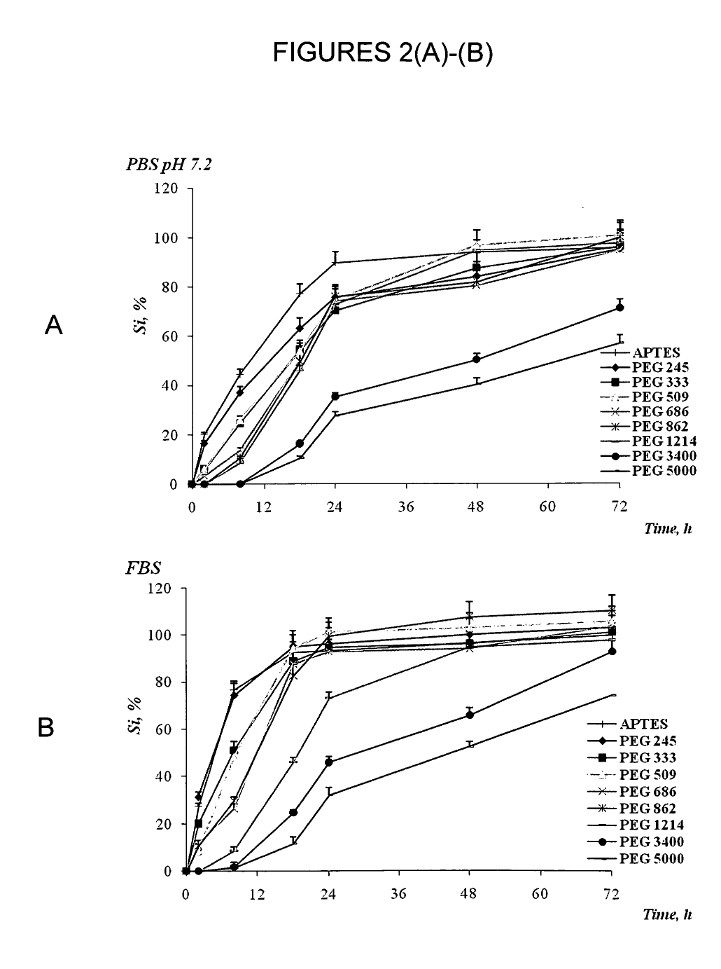

As the following step, the influence of a modification with various PEGs on the degradation kinetics of the particles was evaluated. Seven PEGs with varying molecular weights were employed: 245, 333, 509, 686, 1214, 3400 and 5000 Da. FIGS. 2A-B show degradation profiles of large pores PEGylated particles in PBS and 100% serum in vitro at 37° C.

Generally, particles degraded faster in serum, and the higher was the PEG's molecular weight, the slower was the degradation profile of the particles in both physiological media. The conjugation of the PEG with lowest molecular weight to the porous material's surface did not induce any change in the degradation kinetics in serum, but inhibited degradation and consequently the release of orthosilicic acid into buffer. When PEGs with the longer chains were evaluated, Si mass loss from the particles was slowed, and they almost fully degraded within 18 to 24 hours in serum and within 48 hours in PBS.

The most dramatic effect was observed for PEGs 3400 and 5000 which inhibited the degradation of the systems very prominently, with complete degradation achieved after four days. For these particles during the early stages of the degradation, there was a “lag” period of little or no mass loss.

The degradation process as a function of time as shown in FIG. 2 A-B, can be separated into two phases: phase I, up to about 24 hours; and phase II, from 24 hours onward. The percentage of Si released (Mₜ) in solution over time can be described quite accurately in both phases employing a general power law αt^{β} with different scaling coefficients. Regarding the phase I, the APTES modified surface and short PEG chains (PEG245) behave similarly with Mₜ growing with time following a square root relationship (Mₜ=α\sqrt{t}) with α=23.10 and 23.48 (R²=0.965 and 0.984 as from Table 2), respectively.

TABLE 2

| | Scaling coefficients | | R² |
|---|---|---|---|
| APTES | α = 23.10 | β = 0.5 | 0.965 |
| PEG 245 | α = 23.48 | β = 0.5 | 0.984 |
| PEG 333 | α = 11.81 | β = 0.7 | 0.999 |
| PEG 509 | α = 7.03 | β = 0.9 | 0.994 |
| PEG 686 | α = 2.02 | β = 1.3 | 0.986 |
| PEG 862 | α = 1.46 | β = 1.4 | 0.986 |
| PEG 1214 | α = 0.94 | β = 1.5 | 0.998 |
| PEG 3400 | α = 0.0047 | β = 3.5 | 0.999 |
| PEG 5000 | α = 0.0020 | β = 3.0 | 0.999 |

For coating made with longer PEG chains, the exponent β grows with the length of the polymer as listed in Table 2, with β ranging from 0.7 to 1.5; whereas α decreases leading to longer degradation times. Higher-order degradation laws with Mₜ=αt³ have been observed for PEG3400 and PEG5000 with α=0.0047 and α=0.0020, respectively with R²=0.999 in both cases as from Table 2. For phase II, only particles coated with PEG3400 and PEG5000 exhibit a significant degradation, whereas APTES modified and particles with short PEG chains (up to PEG1214) have almost fully degraded after 18 hours. For PEG3400 and PEG5000, the degradation law can be again described through a general power law of the type αt^{β} with β=0.6 and α=6.87 (R²=0.971) and α=5.50 (R²=0.992), respectively.

Surprisingly, for APTES modified and PEG245 coated particles, the degradation laws exhibit a square root behavior, which may be possibly associated with a diffusive release of silicic acid from the porous silicon matrix into the surrounding solution. As the length of the PEG chains attached on the particle surface increases, the diffusion of the silicic acid from the pores, where most of the degradation occurs, to the surrounding media can be more and more hindered possibly by surface steric interactions with the polymer chains. Notably, a similar behavior is observed for PEG3400 and PEG5000 during phase II, with degradation laws exhibiting an exponent β=0.6, which is very close to that associated with pure diffusion (β=0.5). This can suggest that, during phase II, most of the PEG chains decorating the particle surface have been removed and released in the surrounding medium because of the degradation of the first porous layers.

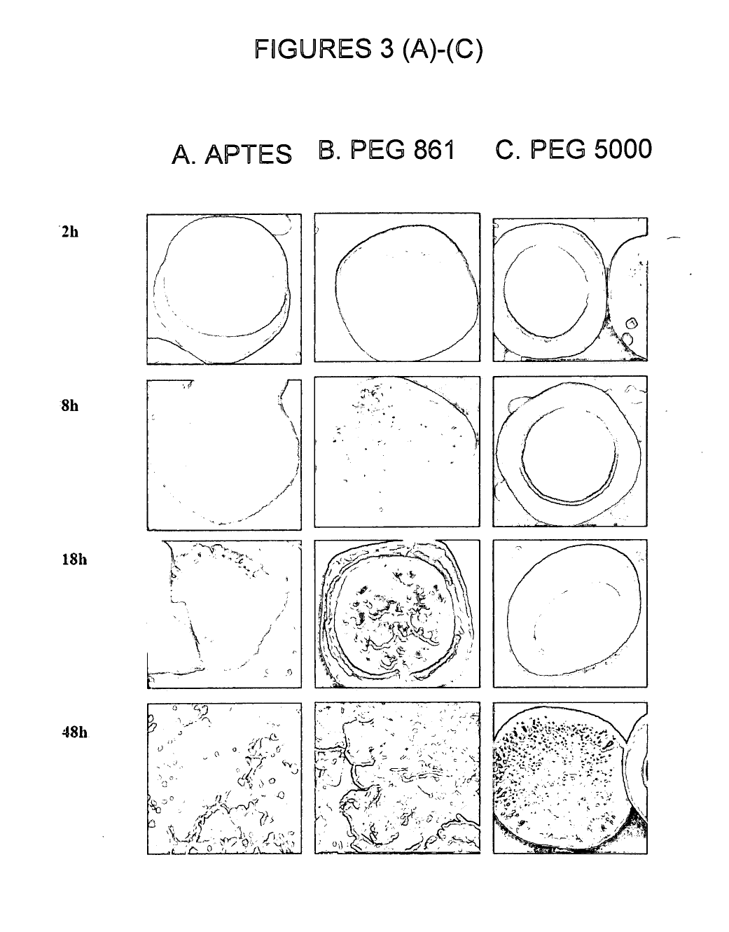

The deterioration of the pSi microparticle surface morphology over time was evaluated by Scanning Electron Microscopy (SEM). FIGS. 3A-C presents SEM micrographs of the particles during the degradation process. The rate of deterioration of the microparticles was associated with the rate of Si chemical degradation, and microparticles conjugated to higher molecular weight PEGs exhibiting surface deterioration at a much slower rate. It can be seen that the degradation of the APTES modified (non-PEGylated) particles over time occurred by means of erosion of the particles surface as well as of the pores. As the study progressed, the pore sizes became wider and the surface of the particle less smooth and more irregular.

With intermediate PEG (MW 861), the appearance of the particles during the in vitro degradation process changed. The most prominent erosion can be seen in the pores in comparison to the particle's outer surface. Although the present inventions are not limited by a theory of operation, this different degradation pattern can be attributed to the steric hindrance of the hydrophilic polymer molecules, which probably can cover particle surface more efficiently outside the pores, thus preventing penetration of water and other components, which play an important role in the degradation process.

In the case of high MW PEG (5000) almost no degradation can be seen within the first 48 hours, which can confirm the data obtained by ICP-AES analysis.

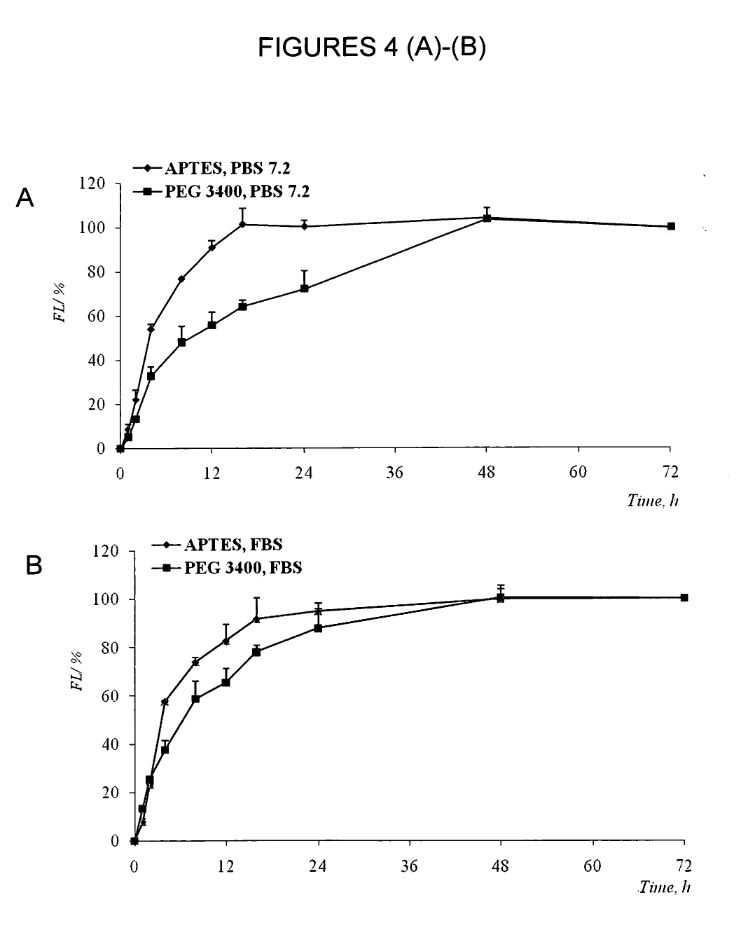

To evaluate the kinetics of surface degradation of the particles, APTES and PEG3400 particles were labeled with the Dylight 488 fluorescent probe. The release kinetics of the probe from particles surface into the degradation media was followed by fluorescence intensity and FACS. Based on the fluorometric analysis, for non-PEGylated particles, the fluorescent probe conjugated to the surface was released into the degradation medium within 8-16 hours depending on the degradation medium. For PEGylated particles the surface erosion rate was significantly extended and the fluorescent probe was released from the particle surface only after 24-48 hours (FIGS. 4A-B). The obtained profiles were in agreement with the data on degradation kinetics of the particles surface as evaluated by ICP-AES and SEM.

The ability to control the release of drug (therapeutic agents) and imaging agents from pharmaceutical systems can be critical for many clinical applications. In the case of the multistage delivery carrier^{[6]} comprising 1^{st} stage microparticles containing 2^{nd} stage nanoparticles within the pores of pSi, the release of the 2^{nd} nanoparticles from the 1^{st} stage pSi microparticles can depend on several mechanisms, including their diffusion outside the pores, as well as on the simultaneous Si erosion and degradation of the matrix. The mechanism of degradation and drug release from biodegradable controlled release systems can generally be described in terms of three basic parameters. First, the type of the hydrolytically unstable linkage in the system and its position. Second, the way the system biodegrades, either at the surface or uniformly throughout the matrix, can affect device performance substantially. The third significant factor can be the design of the drug delivery system encountering for system geometry and morphology as well as for the mechanism of loading of therapeutic agents. For example, the active agent may be covalently attached to the particle matrix and released as the bond between drug and polymer cleaves.

The size and number of pores in porous Si can affect its physiochemical properties, and as a consequence different types of mesoporous Si particles can degrade in aqueous solutions and biological fluids at different rates. The pores of the particles can be considered as a void fraction, being in constant contact with the degradation fluids and presumably originating the orthosilicic acid—the degradation product of porous silicon. Orthosilicic acid, Si(OH)4, is the biologically relevant water soluble form of silicon (Si), recently proven to be play a significant role in bone and collagen growth. Porous Si films can release Si(OH)4 (silicic acid) in aqueous solutions in the physiological pH range through hydrolysis of the Si—O bonds, ^{[16]} which can harmlessly excreted in the urine through the kidneys.^{[17]} The present study addressed the question how the surface modification of pSi surface with PEG can affect the degradation kinetics. APTES particles are a subject of homogenous surface degradation, where the erosion occurs homogeneously throughout the whole surface of the particle as well as the pores. In the case of PEGylated Si particles, the obtained degradation profile can be defined as heterogeneous erosion which besides the surface area, geometry and morphology of the particles is also defined by the length of the polymer chains covering the particle surface. PEGylation in this case can be the factor which controls penetration of solutes into the Si matrix of the particles.

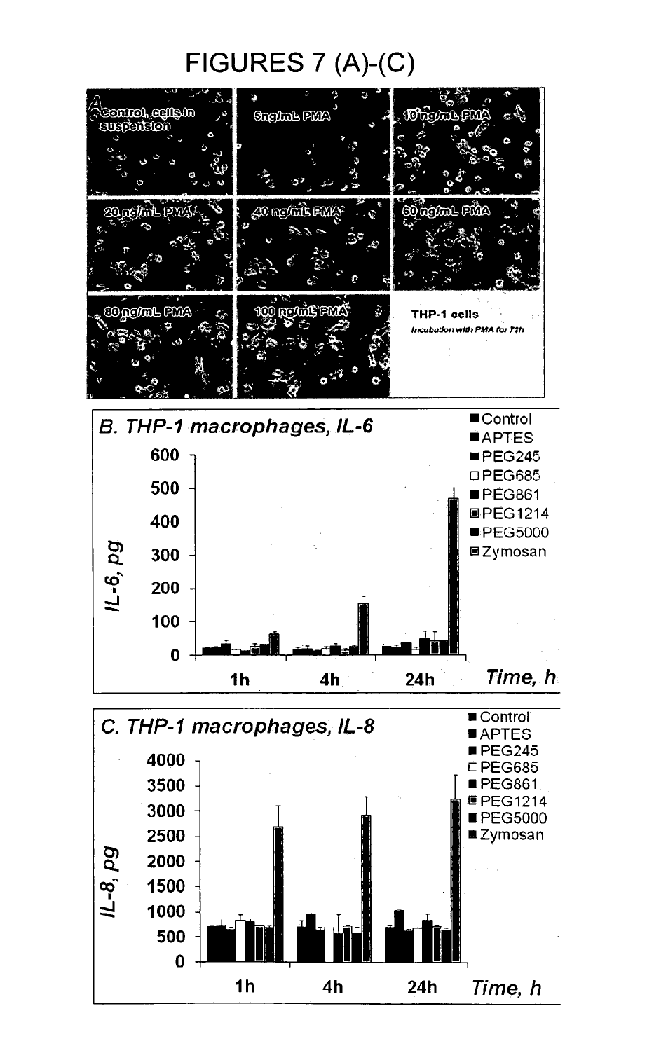

Events that follow the administration of foreign material into the body can provoke acute or chronic inflammation, while the last one can be characterized by the presence of macrophages and release of inflammatory cytokines. Injectable biomaterials are expected to be biocompatible in terms of lack of immunogenic and inflammatory responses. Though silicon has been recognized as an essential trace element in the body which participates in connective tissue, especially cartilage and bone formation, ^{[19]} some forms of crystalline silicon dioxide are known as a cytotoxic agent in macrophages. ^{[20, 21]} Thus, it is important to assess the effect of pSi microparticles with various surface modifications on human immune cells. Keeping this in mind, the biocompatibility of the systems with human monocyte derived differentiated cultured macrophages was evaluated. Data clearly demonstrate that the tested systems did not induce release of proinflammatory cytokines IL-6 and IL-8 over 48 hours period time in THP-1 macrophages (FIGS. 7(A)-(C)). On contrary, when the cells were incubated with zymosan particles, a positive control, a very prominent increase in the cytokines release was observed. Phagocytic receptors on macrophages bind zymosan, stimulate particles engulfment and cytokines release. This agent is well known to induce inflammatory signals in macrophages through toll-like receptors TLR2 and TLR6.

A fine control of the degradation and release kinetics of mesoporous silicon structures can be of fundamental importance in the development of multistage and multifunctional delivery systems. pSi microcarriers can be administered systemically and used to deliver the payload of different nature (therapeutic agents, imaging agents). The size of the pores and the surface chemistry of the pSi structure can be controlled during the fabrication process and thereafter.

It was found that by conjugation of PEGs with various backbone length to porosified silicon microparticles, it is possible to finely tune the degradation kinetics of the material possessing large pores size. The most dramatic effect was observed for PEGs 3400 and 5000 which inhibited the degradation of the systems over more than 3 days. These data point toward the possibility to control degradation of mesoporous silicon microparticles and devices by means of PEGylation and have important clinical implications.

### EXPERIMENTAL

### Fabrication, Surface Modification and Characterization of Porous Silicon Particles

Mesoporous silicon microparticles were fabricated by photolithography and electrochemical etching in the Microelectronics Research Center at The University of Texas at Austin as previously described [6]. The large pore (LP, 30-40 nm pores) silicon particles were formed in a mixture of hydrofluoric acid (49% HF) and ethanol (3:7 v/v) by applying a current density of 80 mA cm⁻² for 25 s. A high porosity layer was formed by applying a current density of 320 mA cm⁻² for 6 s. For fabrication of small pores (SP, 10 nm) silicon particles, a solution of HF and ethanol was used with a ratio of 1:1 (v/v), with a current density applied of 6 mA cm⁻² for 1.75 min. After removing the nitride layer by HF, particles were released by ultrasound in isopropyl alcohol (IPA) for 1 min.

Silicon microparticles in IPA were dried in a glass beaker by heating (80-90° C.) and then oxidized in a piranha solution (1:2 H₂O_{2:}concentrated H₂SO₄ (v/v)) at 100-110° C. for 2 h, with intermittent sonication to disperse the aggregates, washed in DI water and stored at 4° C. in DI water until further use. Prior to modification with 3-Aminopropyltriethoxysilane (APTES, Sigma). The particles were then washed with DI water followed by IPA, suspended in IPA containing APTES (0.5% v/v) for 45 min at room temperature, washed 5 times with IPA and stored in IPA at 4° C.

APTES modified large pore particles were reacted with 10 mM mPEG-SCM or NHS-m-dPEG in 400-500 μl acetonitrile for 1.5 hours. The succinimidyl ester on the PEGs reacts with an amino group that is exposed on the surface of the APTES particles giving a stable chemical linkage of PEGs to the particles. The particles were then washed (by centrifugation) in deionized water 4-6 times to remove any unreacted PEGs. The particles were stored in deionized water or IPA at 4 ° C. till further use.

Volumetric particle size, size distribution and count was obtained using a Z2 Coulter® Particle Counter and Size Analyzer (Beckman Coulter, Fullerton, Calif., USA). Prior to the analysis, the samples were dispersed in the balanced electrolyte solution (ISOTON® II Diluent, Beckman Coulter Fullerton, Calif., USA) and sonicated for 5 seconds to ensure a homogenous dispersion.

The zeta potential of the silicon particles was analyzed using a Zetasizer nano ZS (Malvern Instruments Ltd., Southborough, Mass., USA). For the analysis, 2μL particle suspension containing at least 2×10⁵ particles to give a stable zeta value evaluation were injected into a sample cell countering filed with phosphate buffer (PB, 1.4 mL, pH 7.3). The cell was sonicated for 2 min, and then an electrode-probe was put into the cell. Measurements were conducted at room temperature (23° C.) in triplicates.

### Degradation Study in Simulated Physiological Conditions

To evaluate degradation kinetics, 10⁷ of the particles were added to PBS (1.5 mL, pH 7.2) or 100% fetal bovine serum (FBS). The samples (n=3) were incubated at 37 C and constantly mixed using a rotary shaker until the appropriate time points had elapsed. Aliquots (85 μL) were taken from the tubes: 75 μL were filter-spun (0.45 μm filter) to separate the undegraded particles from the degradation medium and the resulting liquid was stored at 4° C. for later analysis of total silicon by Inductively Coupled Plasma Atomic Emission Spectrometer (ICP-AES). The remaining 10 μL were extensively washed with deionized (DI) water to remove the salts, placed on the grid, dried in a dessicator and further analyzed for particles morphology by Scanning Electron Microscopy (SEM). In the case of fluorescent PEG conjugated to the surface of the particles, the samples (150 μL) were spun down at 4500 rpm×20 min, the supernatant was collected into 96 wells plates and analyzed for the quantity of fluorophore released from the particles by fluorimetry and for Si contents by ICP-AES. Silicon contents released to from the particles during the degradation process was measured using a Varian Vista-Pro ICP-AES. Si was detected at 250.69, 251. 43, 251.61 and 288.158 nm. A calibration run including the internal control (Yttrium, 1 ppm) was made before each group of 1 w sample (100%), the particles were dissolved in 1N NaOH for 4 hours in 37C. Further, all results were expressed as % of the silicic acid released to the medium. SEM was applied to examine the structure and morphology of the particles. Samples were sputter-coated with gold for 2 min at 10 nm using a CrC-150 Sputtering System (Torr International, New Windsor, N.Y.) and observed under a FEI Quanta 400 field emission scanning electron microscope (FEI Company, Hillsboro, Oreg.) at an accelerating voltage of 20 kV, chamber pressure of 0.45 Torr and spot size 5.0.

Fluorescence of the particles conjugated to FITC-PEG (MW 3400) was assessed using a FACScalibur (Becton Dickinson). Bivariate dot-plots defining logarithmic side scatter (SSC) versus logarithmic forward scatter (FSC) were used to evaluate the size and shape of the unlabeled silicon particles (3 μm in diameter, 1.5 μm in height) and to exclude non-specific events from the analysis. A polygonal region (R1) was defined as an electronic gate around the centre of the major population of interest for undegraded particles, which excluded events that were too close to the signal-to-noise ratio limits of the cytometer. The peaks identified in each of the samples were analyzed in the corresponding fluorescent histogram and the geometric mean values recorded. For particle detection, the detectors used were FSC E-1 and SSC with a voltage setting of 474 volts (V). The fluorescent detector FL1 was set at 800 V and green fluorescence was detected with FL1 using a 530/30 nm band-pass filter. For each analysis, 50,000-200,000 gated events were collected. Instrument calibration was carried out before, in between, and after each series of experiments for data acquisition using BD Calibrite™ beads (3.5 μm in size).

For the analysis of fluorescence intensity analysis the samples were placed on a 96-well plate (Nunclon, Denmark) and quantities of FITC-PEG released from the particles surface were determined in triplicates using BMG FluoSTAR microplate variable wavelength fluorescence spectrophotometer (Galaxy, excitation 488 nm, emission 523 nm).

Based on the observed experimental results, a mathematical model was identified and used to get further insight into the underlying physical and chemical processes which are involved in the effect of PEGylation on particles degradation.

### Evaluation of Biocompatibility of PEGylated Particles with Human Macrophages in vitro

THP-1 monocyte cell line was obtained from the American Type Culture Collection (Manassas, Va.). Cells were cultured at 0.4-2×10⁶ cells/mL in RPMI 1640 containing heat-inactivated FCS (10% w/v), glutamine (2 mM), penicillin (100 U/mL), and streptomycin (100 μg/mL), and maintained at 37° C. under 5% CO2. All reagents and medium were purchased from ATCC and Gibco BRL (Gaithersburg, Md.). THP-1 cells (0.2×10⁶ cells/well) were differentiated into macrophages in 24 well plates containing 1 mL medium/well with phorbol ester (80 ng, PMA, Sigma USA) over 72 h. A stock solution of PMA was prepared by dissolving PMA in sterile dimethylsulfoxide (Sigma). The stock solution was stored frozen at −20° C. Immediately prior to use, the PMA stock solution was diluted in RPMI medium. The differentiation-inducing dose of PMA for THP-1 cells was determined in preliminary dose-response experiments (data not shown). The criteria for differentiation of THP-1 cells were cell adherence, changes in cell morphology, and changes in the cell surface marker expression profile. Following 72 hours incubation, the cells were washed two times with the medium and incubated with particles (5 particles/cell). The supernatants were collected and stored at −70 ° C. until the cytokine analysis. Proinflammatory cytokines, interleukin-6 (IL-6) and interleukin-8 (IL-8) were analyzed using commercial ELISA kits (BD Biosciences).

### REFERENCES

[1] A. Uhlir, Bell System Tech. J., 1956, 35, 333-347.

### [2] A. J. Nijdam, M. C. Cheng, D. H. Geho, R. Fedele, P. Herrmann, K. Killian, V. Espina, E. F. Petricoin, L. A. Liotta, M. Ferrari, Biomaterials, 2007, 28, 550-558.

### [3] V. S. Lin, K. Motesharei, K. P. Dancil, M. J. Sailor, M. R. Ghadiri, Science, 1997, 278, 840-843

[4] V. Lehmann, U. Gosele, Appl. Phys. Lett., 1991, 131, 58.

[5] Y. Y. Li, F. Cunin, J. R. Link, T. Gao, R. E. Betts, S. H. Reiver, V. Chin, S. N. Bhatia, M. J. Sailor, Science, 2003, 299, 2045-2047.

[6] E. Tasciotti, X. Liu, R. Bhavane, K. Plant, A. Leonard, B. Price, M.C. Cheng, P. Decuzzi, J. Tour, F. Robertson, M. Ferrari, Nat. Nanotech., 2008, 3, 151-157.

[7] S. Sharma, A. J. Nijdam, P. M. Sinha, R. J. Walczak, X. Liu, M. C. Cheng, M. Ferrari, Expert Opin. Drug Deliv., 2006, 3, 379-394.

[8] J. Salonen, L. Laitinen, A. M. Kaukonen, J. Tuura, M. Björkqvist, T. Heikkila, K. Vähä-Heikkilä, J. Hirvonen, V. P. Lehto, J. Control. Rel., 2005, 108, 362-374.

[9] M. V. Brumm, Q. D. Nguyen, Int. J. Nanomedicine., 2007, 2, 55-64.

[10] A. S. Goh, A. Y. Chung, R. H. Lo, T.N Lau, S. W. Yu, M. Chng, S. Satchithanantham, S. L. Loong, D. C. Ng, B. C. Lim, S. Connor, P. K. Chow, Int. J. Radiat. Oncol. Biol. Phys., 2007, 67, 786-92.

[11] L. T. Canham, C. L. Reeves, J. P. Newey, M. R. Houlton, T. I. Cox, J. M. Buriak, M. P. Stewart, Adv. Mater., 1999, 11, 1505-1507.

[12] L. T. Canham, Adv. Mat., 1995, 7, 1033-10371.

[13] M. L. Immordino, F. Dosio, L. Cattel, Int. J. Nanomedicine, 2006, 1, 297-315.

[14] A. P. Chapman. Adv. Drug Deliv. Rev., 2002, 54, 531-545.

[15] U. Wattendorf, H. P. Merkle, J. Pharm. Sci., 2008, published online.

[16] H. Böttcher, P. Slowik, W. Süss, J. Sol-Gel Sci. Technol., 1998, 13, 277-281.

[17] D. M. Reffitt, R. Jugdaohsingh, R. P. H. Thompson, J. J. Powell, J. Inorg. Biochem., 1999, 76, 141-147.

[18] E. M. Carlisle, Science, 1970, 167, 279-280.

[19] M. P. Absher, L. Trombley, D. R. Hemenway, R. M. Mickey, K. O. Leslie, Am. J. Pathol., 1987, 134, 1243-1251.

[20] J. M. Buriak, M. J. Allen, J. Am. Chem. Soc., 1998, 120, 1339.

[21] V. Kolb-Bachofen, J. Clin. Invest., 1992, 90, 1819-1824.

[22] J. Wilson, G. H. Pigott, F. J. Schoen, L. L. Hench, J. Biomed. Mater. Res., 1981, 15, 805-817.

[23] G. Palumbo, L. Avigliano, G. Strukul, F. Pinna, D. del Principe, I. d'Angelo, M. Anniccjiarico-Petruzzelli, B. Locardi, N. Rosato, J. Mat. Sci. Mat. Med, 1997, 8, 417-421.

[24] V. Chin, B. E. Collins, M. J. Sailor, S. N. Bhatia, Adv. Mater., 2001, 13, 1877-1880.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A biodegradable object, comprising a porous body, that has an outer surface, and polymer chains disposed on said outer surface, wherein biodegradation kinetics of the object is determined by a pore size in the porous body and a molecular weight of the polymer chains.

2. The object of claim 1, wherein said object comprises a plurality of microparticles or nanoparticles.

3. The object of claim 1, that is an implant.

4. The object of claim 1, wherein the porous body comprises a porous etched material.

5. The object of claim 4, wherein the porous body comprises porous silicon.

6. The object of claim 1, wherein the porous body comprises a nanoporous material.

7. The object of claim 1, wherein the polymer chains are hydrophilic polymer chains.

8. The object of claim 1, wherein the polymer chains comprise polyethylene glycol.

9. The object of claim 1, wherein the polymer chains are covalently bound to the outer surface.

10. The object of claim 1, wherein the porous body has a pore size from 25 to 120 nm.

11. The object of claim 10, wherein the porous body has a pore size from 30 to 60 nm.

12. The object of claim 10, wherein the polymer chains have a molecular weight from about 800 to about 10,000.

13. The object of claim 10, wherein the polymer chains have a molecular weight from about 800 to about 7,000.

14. The object of claim 1, that is biocompatible.

15. The object of claim 1, further comprising an active agent in pores of the porous body.

16. The object of claim 15, wherein the active agent comprises a therapeutic agent.

17. The object of claim 15, wherein the active agent comprises an imaging agent.

18. The object of claim 1, that has a heterogeneous biodegradation profile.

19. A method of making a biodegradable object comprising
A) obtaining an object, that has a porous body and an outer surface, wherein a biodegradation time i) is determined by a pore size of the porous body and ii) is less than a desired biodegradation time value; and
B) modifying the biodegradation time of the object to the desired biodegradation time value by disposing on the outer surface of the object polymer chains, wherein the modified biodegradation time of the object is determined by the pore size of the porous body and a molecular weight of the polymer chains.

20. The method of claim 19, wherein the porous body of the object comprises a porous etched material.

21. The method of claim 20, wherein the porous body of the object comprises porous silicon.

22. The method of claim 19, wherein the porous body of the object comprises a nanoporous material.

23. The method of claim 19, wherein the polymer chains are hydrophilic polymer chains.

24. The method of claim 23, wherein the polymer chains are polyethylene glycol chains.

25. The method of claim 19, wherein the porous body has a pore size from 25 to 120 nm.

26. The method of claim 25, wherein the polymer chains have a molecular weight from about 800 to about 10,000.

27. The method of claim 25, wherein the polymer chains have a molecular weight from about 800 to about 7,000.

28. The method of claim 19, wherein after said disposing the object has a heterogeneous biodegradation profile.

29. The method of claim 19, further comprising loading an active agent in pores of the porous body of the object.

30. The method of claim 19, wherein said object is an implant.

31. The method of claim 19, wherein said object comprises a plurality of micro or nanoparticles.

32. The method of claim 19, wherein said disposing comprises covalently binding the polymer chains to the outer surface.

33. A delivery method comprising introducing into a body of a subject a biodegradable object made according to the method of claim 19.

34. The method of claim 33, wherein said introducing comprises intravascularly injecting said object in the subject.

35. The method of claim 34, wherein said introducing comprises implanting said object in the subject.

36. A delivery method comprising
introducing into a body of a subject a biodegradable object that comprises a porous body, an outer surface and polymer chains disposed on said outer surface, wherein biodegradation kinetics of the object is determined by a pore size in the porous body and a molecular weight of the polymer chains.

37. The method of claim 36, wherein said object comprises a matrix of micro or nanoparticles.

38. The method of claim 36, wherein and said introducing comprises intravascularly injecting said object in the subject.

39. The method of claim 36, wherein said object comprises an implantable device and said introducing comprises implanting said object in the subject.

40. The method of claim 36, wherein the porous body comprises a porous etched material.

41. The method of claim 40, wherein the porous body comprises porous silicon.

42. The method of claim 36, wherein the porous body comprises a nanoporous material.

43. The method of claim 36, wherein the polymer chains are hydrophilic polymer chains.

44. The method of claim 43, wherein the polymer chains are polyethylene glycol chains.

45. The method of claim 36, wherein the polymer chains are covalently bound to the outer surface.

46. The method of claim 36, wherein the porous body has a pore size from 25 to 120 nm.

47. The method of claim 46, wherein the porous body has a pore size from 30 to 50 nm.

48. The method of claim 46, wherein the polymer chains have a molecular weight from about 800 to about 10,000.

49. The method of claim 48, wherein the polymer chains have a molecular weight from about 800 to about 7,000.

50. The method of claim 36, wherein the object further comprises an active agent in pores of the porous body.

51. The method of claim 50, wherein the active agent comprises a therapeutic agent.

52. The method of claim 50, wherein the active agent comprises an imaging agent.

53. The method of claim 36, wherein the object has a heterogeneous biodegradation profile.

54. The method of claim 37, wherein the subject is a human being.
